(19) United States
(12) Patent Application Publication
(10) Pub. No.: US 2012/0316633 A1
(43) Pub. Date: Dec. 13, 2012

(54) Durable Stent Drug Eluting Coating

(75) Inventors: Aiden Flanagan, Co. Galway (IE); Jan Weber, Maastricht (NL)

(73) Assignee: BOSTON SCIENTIFIC SCIMED, INC., Maple Grove, MN (US)

(21) Appl. No.: 13/489,151

(22) Filed: Jun. 5, 2012

Related U.S. Application Data
(60) Provisional application No. 61/494,169, filed on Jun. 7, 2011.

(51) Int. Cl. A61F 2/06; B05D 1/18; C23C 16/44; B05D 1/02

(52) U.S. Cl. 623/1.11; 623/1.42; 427/2.1

(57) ABSTRACT

In embodiments, medical devices, such stents, can deliver a therapeutic agent to body tissue of a patient. The medical device includes a porous therapeutic layer that is substantially free of a polymer matrix which can withstand expansion or contraction of the medical device, with minimal delamination.

### CROSS-REFERENCE TO RELATED APPLICATION

This application claims priority under 35 USC §119(e) to U.S. Provisional Patent Application Ser. No. 61/494,169, filed on Jun. 7, 2011, the entire contents of which are hereby incorporated by reference.

### TECHNICAL FIELD

The present invention relates to medical devices, and in particular, medical devices that have porous drug coating.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, e.g., so that it can contact the walls of the lumen. Stent delivery is further discussed in Heath, U.S. Pat. No. 6,290,721, the entire content of which is hereby incorporated by reference herein. The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn from the lumen.

### SUMMARY

Therapeutic agents can be delivered to body lumens via endoprostheses. The present disclosure is based, at least in part, on a drug eluting endoprosthesis having a coating of therapeutic agent that is flexible and adherent to the endoprosthesis surface. The coating can be substantially free of a polymer matrix and can be coated on medical devices such as stents, balloons, pacing leads, vascular closing devices, etc.

Accordingly, in one aspect, the disclosure features an expandable medical device including a porous substantially polymer-free coating including a therapeutic agent. The coating substantially adheres to the medical device upon expansion of the medical device.

In another aspect, the disclosure features a method of making a medical device. The method includes step (a): forming a mixture including a therapeutic agent, an organic solvent, and optionally water; step (b): providing a solution including water, when the mixture in step (a) is water-free; step (c): coating the medical device with the mixture and the solution, when present; and step (d): evaporating the organic solvent and water to provide a porous coating including a therapeutic agent.

In a further aspect, the disclosure features a method of making a medical device. The method includes step (a): forming a mixture including a hydrophilic therapeutic agent and water; step (b): providing a solution including a solvent having a higher boiling point than water; step (c): coating the medical device with the mixture and the solution; and step (d): evaporating the water and solution to provide a porous coating including a hydrophilic therapeutic agent.

In yet a further aspect, the disclosure features a method of making a medical device. The method includes step (a): forming a mixture comprising a therapeutic agent, an organic solvent, and water; step (b): ultrasonicating the mixture to provide a dispersion; step (c): coating the medical device with the dispersion; and step (d): evaporating the water and organic to provide a porous coating comprising a therapeutic agent.

Embodiments of the above-mentioned medical devices can have one or more of the following features.

In some embodiments, the coating further includes aluminum oxide, titanium oxide, tin oxide, zinc oxide, or silica. The coating can have a porosity of about 20% or more. The porous substantially polymer-free coating can consist essentially of one or more therapeutic agents.

In some embodiments, the expandable medical device includes a stent, a balloon, a balloon catheter, a self-expanding stent and a delivery catheter.

In some embodiments, the therapeutic agent is hydrophobic. The therapeutic agent can include paclitaxel, everolimus, rapamycin, sirolimus, and/or tacrolimus. In some embodiments, the therapeutic agent is hydrophilic. The therapeutic agent can include heparin, diclofenac, and/or aspirin. The therapeutic agent can be amorphous. The porous coating can substantially adhere (e.g., be more than about 95% adherent) to the medical device upon expansion or contraction of the medical device. When inserted to a predetermined location in a blood vessel, about 50% or more of the therapeutic agent can be released from the coating in about 10 days or less.

In some embodiments, step (c) further includes simultaneously coating the medical device with the mixture and the solution, when present. Prior to evaporation, the ratio of organic solvent to water on the medical device can be about 1:1 or greater. Coating the medical device can include spraying (e.g., spray-coating) and/or dip-coating the medical device. The mixture, which can include a therapeutic agent, an organic solvent, and optionally water, can further include a polymer. In some embodiments, the solution can further include a polymer. In some embodiments, the method can further include step (e): coating the medical device with aluminum oxide, titanium oxide, tin oxide, zinc oxide, and/or silica. Step (e) can include coating the medical device using atomic layer deposition, and can precede step (c) or follow step (d). In some embodiments, the method further includes repeating one or more of steps (a), (b), (c), (d), or (e).

In some embodiments, when the method includes coating the medical device with the dispersion, the method can further include step (e) before step a: applying a porous polymer coating onto the medical device. The method can further include step (f) after step (e) and before step (a), or after step (d): coating the medical device with aluminum oxide, titanium oxide, tin oxide, zinc oxide, and/or silica.

Embodiments and/or aspects can provide one or more of the following advantages.

In some embodiments, a porous drug-eluting coating can provide enhanced flexibility compared to a solid coating. The porous coating can be more adherent to an underlying substrate, compared to a solid coating. The porous coating can be substantially free of a polymer matrix and can minimize inflammatory responses when a coated medical device is inserted and/or implanted in a body lumen. The porous coating can be relatively easy to make. In some embodiments, a medical coated with a porous coating can be relatively durable. The porous coating can be robust. For example, the porous coating can remain substantially intact (e.g., more than about 95% intact, more than about 98% intact, more than about 99% intact) as a coated medical device is inserted and/or implanted in a body lumen. In some embodiments, a porous coating that is coated with an elution control membrane is more effective at delaying the drug elution. For example, a porous coating can create a more tortuous path so as to delay drug elution, compared to a non-porous coating.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1C are longitudinal cross-sectional views illustrating delivery of a stent in a collapsed state, expansion of the stent, and deployment of the stent;

FIG. 2 is a perspective view of a stent;

FIGS. 3A and 3B are micrographs of a coating on a medical device;

FIG. 4 is a cross sectional view of a medical device;

FIG. 5 is a micrograph of a coating on a medical device;

FIG. 6 is a micrograph of a coating on a medical device;

FIG. 7 is a micrograph of a coating on a medical device;

FIG. 8 is a micrograph of a coating on a medical device;

FIGS. 9A and 9B are micrographs of a coating on a medical device;

FIG. 10 is a micrograph of a coating on a medical device; and

FIG. 11 is a micrograph of a coating on a medical device.

Like reference symbols in the various drawings indicate like elements.

### DETAILED DESCRIPTION

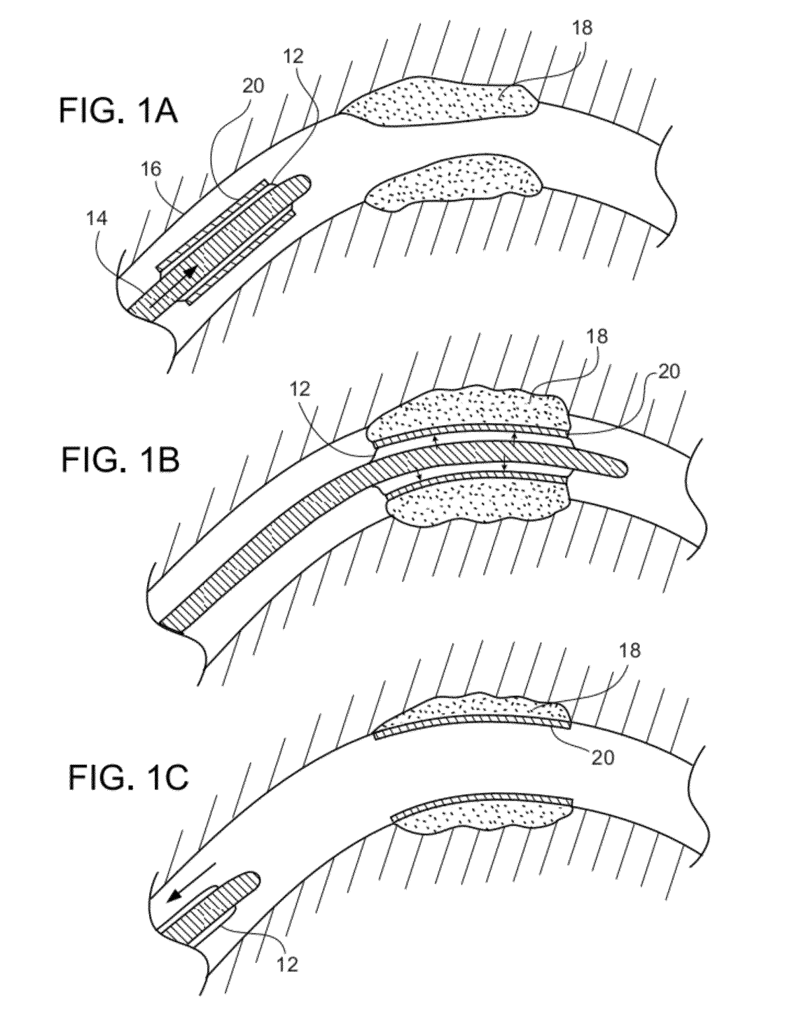

Referring to FIGS. 1A-1C, a stent 20 is placed over a balloon 12 carried near a distal end of a catheter 14, and is directed through the lumen 16 (FIG. 1A) until the portion carrying the balloon and stent reaches the region of an occlusion 18. The stent 20 is then radially expanded by inflating the balloon 12 and compressed against the vessel wall with the result that occlusion 18 is compressed, and the vessel wall surrounding it undergoes a radial expansion (FIG. 1B). The pressure is then released from the balloon and the catheter is withdrawn from the vessel (FIG. 1C).

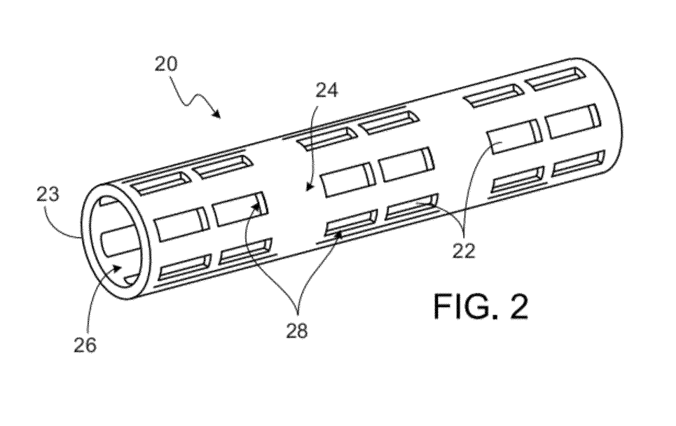

Referring to FIG. 2, an example of one stent 20 includes a plurality of fenestrations 22 defined in a wall 23. Stent 20 includes several surface regions, including an outer, or abluminal, surface 24, an inner, adluminal, surface 26, and a plurality of cutface surfaces 28. The stent can be balloon expandable, as illustrated above, or a self-expanding stent. The stent can have a coating that includes one or more elutable drugs, the coating can cover one or more portions of the stent.

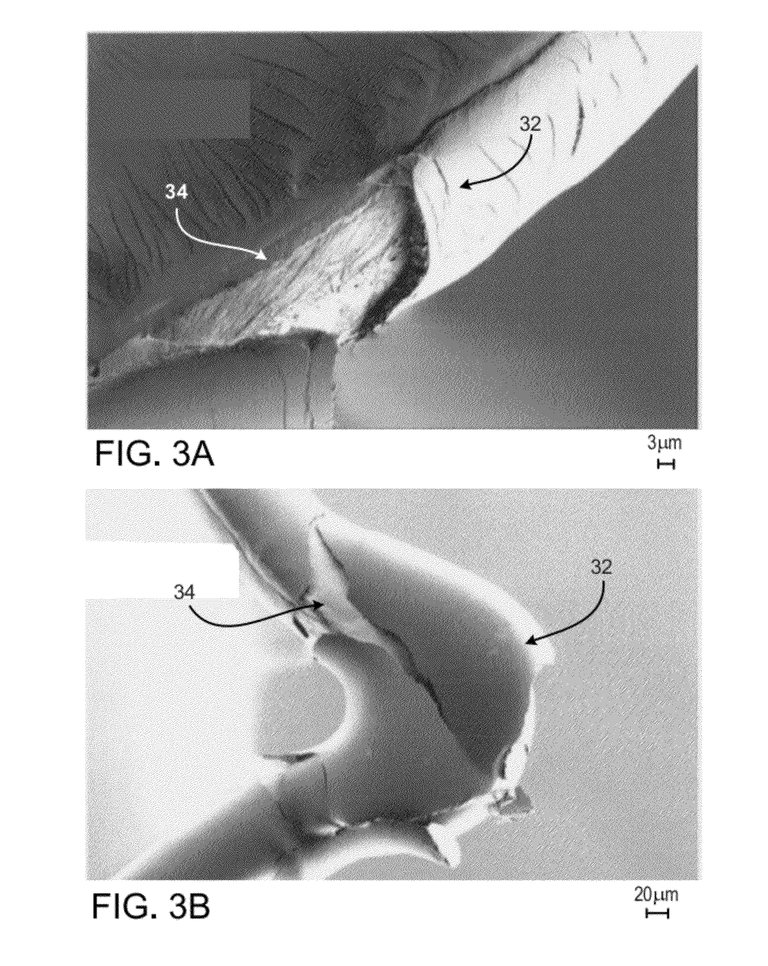

A medical device can include portions that are subjected to bending, stretching, or other deformations during deployment. Referring to FIGS. 3A and 3B, in some embodiments, a solid drug coating 32 including one or more elutable drugs on the surface of these portions can delaminate (e.g., 34) when the medical device is subjected to strain, for example, when a stent is expanded. Such a coating can be brittle and exhibit poor adhesion to the medical device. By providing a porous surface, a porous coating can adhere to the surface of the medical device even when the device is subjected to high strain (e.g., during expansion of the stent). The coating can be substantially free of a polymer matrix and include one or more drugs, which can elute upon insertion of the stent over a desired duration.

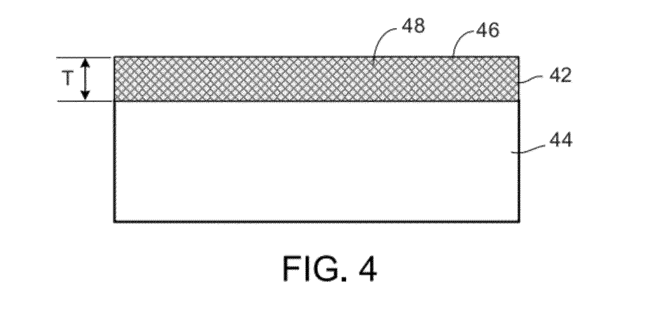

Referring to FIG. 4, a medical device can include a coating 42 over a substrate 44. The coating can be substantially (e.g., about 90% or more, about 95% or more, about 98% or more, about 99% or more, about 100%) formed of one or more therapeutic agents. The coating can be substantially (e.g., about 90% or more, about 95% or more, about 98% or more, about 99% or more, about 100%) free of a polymer matrix (e.g., a polymeric matrix in which the therapeutic agent may be incorporated). As used herein, “about” or “approximately” can refer to a margin of error of ±2% of a given numerical value or ratio. A coating without a polymer matrix can decrease the likelihood of adverse bodily reactions to the polymer matrix and/or or its degradation products. Without a polymer matrix, the drug coating can release a greater amount of drug in a shorter amount of time.

Coating 42 can be porous. A porous coating can allow the coating to compress and stretch without allowing stresses to build up in the coating, which would otherwise cause formation of macrocracks (e.g., a fissure that extends in depth from a coating surface to a medical device surface at greater than about 50% of the fissure length, and that extends over at least half a strut width when the medical device is a stent. For example, the macrocrack can have a length that is greater than about 40 micrometers) and their propagation throughout the coating. In contrast to a macrocrack, a porous coating can have fissures that do not extend in depth to the medical device surface along greater than about 50% of the fissure length, such that the porous coating can maintain its integrity and substantially adhere to the medical device surface. The coating can include a plurality of pores, channels (e.g., interconnecting channels), and voids 46 between solid material 48 such that the coating can have an open structure. The porosity of the coating can be characterized by its percent porosity (“% porosity”), which refers to the ratio of the amount of voids to solid material within the coating. For example, the percent porosity can be a ratio of volume of void to volume of solids—a larger percent porosity indicates a greater amount of voids and lesser amount of solid. A coating can include regions having different percent porosities.

Referring to FIG. 4, in some embodiments, porous coating 42 can have a thickness T of about 0.5 micron or more (e.g., about one micron or more, about two microns or more, about three microns or more, about five microns or more, about 10 microns or more, or about 20 microns or more) and/or about 20 microns or less (e.g., about 10 microns or less, five microns or less, three microns or less, two microns or less, or one micron or less). In some embodiments, the porous coating can have a vol/vol porosity of about 10% or more (e.g., about 20% or more, about 40% or more, about 50% or more, about 60% or more, about 70% or more, about 80% or more) and/or about 90% or less (e.g., about 80% or less, about 70% or less, about 60% or less, about 50% or less, about 40% or less, or about 20% or less). In some embodiments, the porous coating can have a density of about 80% or less (e.g., about 70% or less, about 60% or less, about 50% or less, about 40% or less, about 30% or less, about 20% or less, or about 10% or less) and/or about 10% or more (e.g., about 20% or more, about 30% or more, about 40% or more, about 50% or more, about 60% or more, or about 70% or more) of the density of a solid coating having the same composition. For example, a porous coating can have a density that is about 50% or less that of a solid coating having the same composition. As used herein, a solid coating is a coating having a vol/vol porosity of less than about 10%.

Porosity can be determined by measuring weight and volume of a coating on a device. A greater porosity results when a coating has a smaller weight to volume (or average thickness) ratio (or greater average thickness or volume to weight ratio), and a smaller porosity results when a coating has a larger weight to volume (or average thickness) ratio (or smaller thickness or volume to weight ratio). For example, for identical coating compositions, a solid coating's weight/thickness ratio will be greater than the weight/thickness ratio for a porous coating.

As an example, an average thickness of a coating can be determined by measuring the thickness at several locations (e.g., at least 3, at least 10, or at least 30 locations) of a coated medical device, adding the thicknesses, and dividing the sum by the number of measurements. The approximate surface area of the coating can also be measured by methods known to a person of skill in the art. The coating volume can be calculated from the average thickness and the surface area. The coating density can be obtained by comparing the coating volume to the coating weight, where coating weight is equal to coated device weight minus the bare device weight. Methods of measuring coating thickness are described, for example, in U.S. Pat. Nos. 7,374,791 and 6,764,709, herein incorporated by reference in their entireties. Surface areas can be measured, for example, by measuring a surface area using a Visicon scanning machine.

In some embodiments, by calculating the density of the drug coating, the volume of drug can be calculated to obtain the conventional vol/vol porosity. The ratio of volume of void/volume of drug is calculated as follows:

$$\frac{{M}_{drug}}{{V}_{coat}} = {ρ}_{coat}$$

$${V}_{coat} = {V}_{drug} + {V}_{void} \\ ⇒ {V}_{void} = \frac{{M}_{drug}}{{ρ}_{coat}} - {V}_{drug} ,$$

where ρ_{coat} is the density of the porous coat, M_{drug} is the mass of the drug. V_{void} is the volume (vol) of the void, V_{drug} is the volume (vol) of the drug and:

$${V}_{drug} = \frac{{M}_{drug}}{{ρ}_{drug}} ,$$

where ρ_{drug} is the known solid density of the drug. Hence V_{void}/V_{drug} and the corresponding V_{void}/V_{drug}×100 (vol_{void}/vol_{drug} percent porosity) can be calculated.

In addition to or instead of calculating the porosity by measuring weight and volume, in some embodiments, porosity can be measured using gas/liquid absorption. For example, porosity can be measured using a porosimeter (e.g., a Micromeritics Autopore III porosimeter, available from Micromeritics, Norcross, Ga.). Porosity measurements are described, for example, in Cooper et al., Biomaterials, 26 (2005), 1523-1532.

In some embodiments, a pore dimension can be physically measured from a micrograph image. In some embodiments, one or more pores defined within the coating can have an average volume of from about 0.01 μm³ to 100 μm³. The average volume of the one or more pores can be greater than or equal to about 0.01 μm³ (e.g., greater than or equal to about 0.1 μm³, greater than or equal to about 1 μm³, greater than or equal to about 25 μm³, greater than or equal to about 50 μm³, or greater than or equal to about 90 μm³); and/or less than or equal to about 100 μm³ (e.g., less than or equal to about 90 μm³, less than or equal to about 50 μm³, less than or equal to about 25 μm³, less than or equal to about 1 μm³, or less than or equal to about 0.1 μm³). The one or more pores can also be expressed using an average diameter, such that one or more pores defined within the coating can have an average diameter of from about five μm to about 30 μm. For example, the average diameter of the one or more pores can be greater than or equal to about five μm (e.g., greater than or equal to about eight μm, greater than or equal to about ten μm, greater than or equal to about 12 μm, greater than or equal to about 15 μm, greater than or equal to about 18 μm, greater than or equal to about 20 μm, greater than or equal to about 23 μm, or greater than or equal to about 25 μm); and/or less than or equal to about 30 μm (e.g., less than or equal to about 25 μm, less than or equal to about 23 μm, less than or equal to about 20 μm, less than or equal to about 18 μm, less than or equal to about 15 μm, less than or equal to about 12 μm, less than or equal to about ten μm, or less than or equal to about eight μm). An average diameter is determined by measuring the diameter (e.g., cross-dimension) of a pore at 30 or more locations, and determining the average of these diameter measurements.

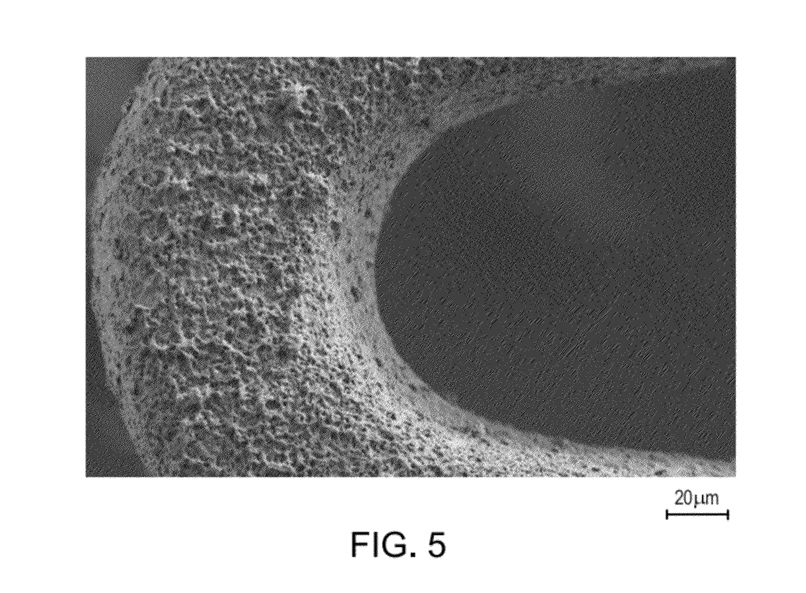

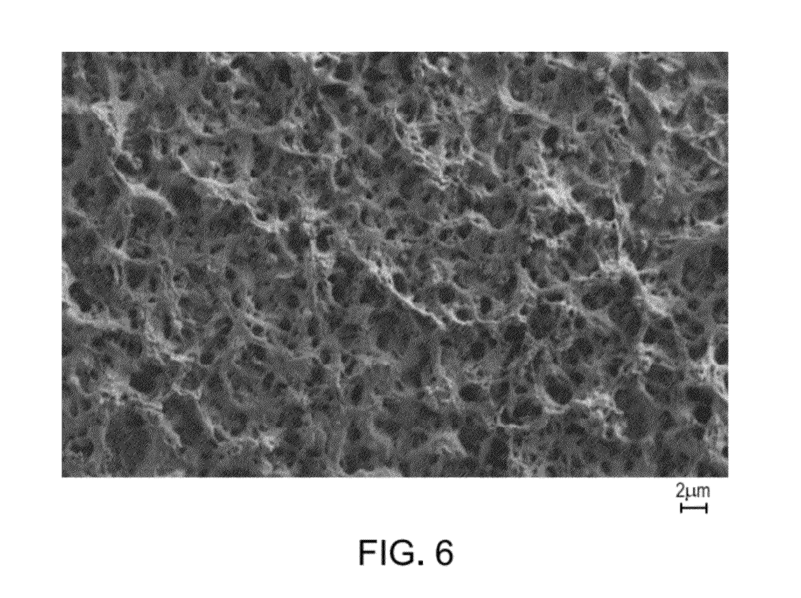

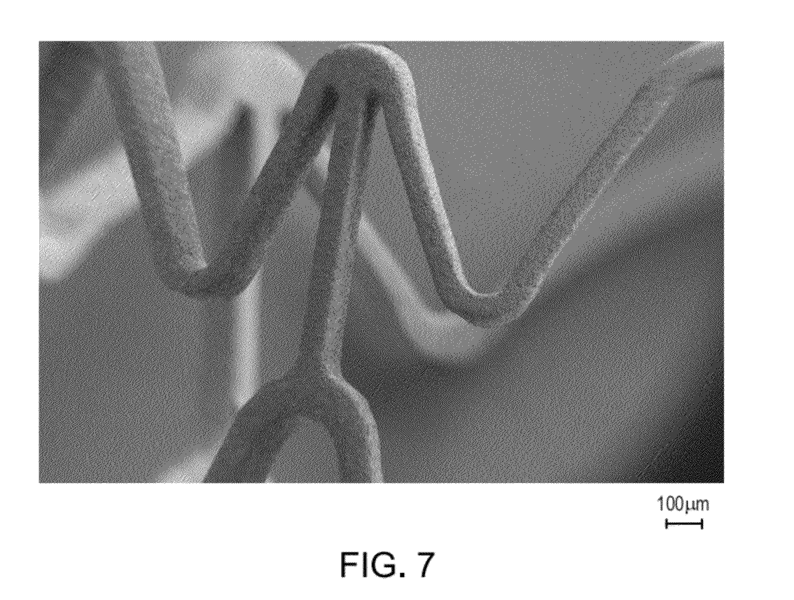

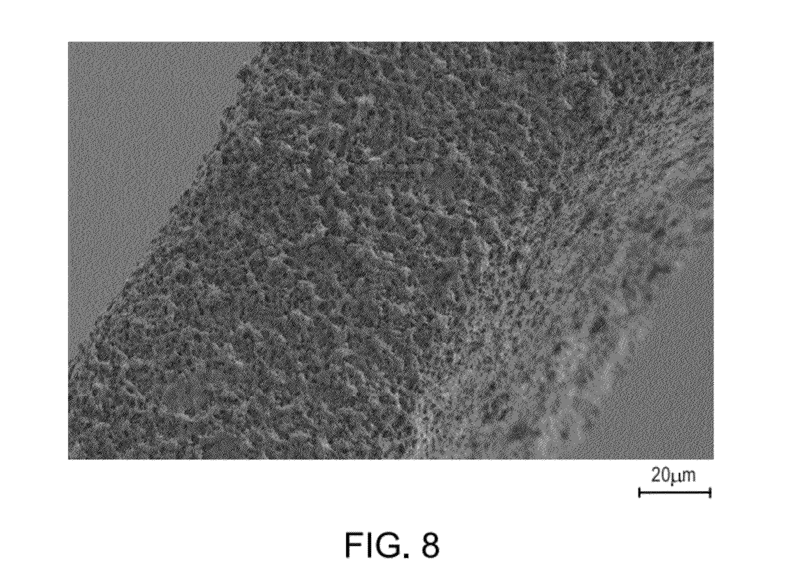

The coating can substantially adhere to the underlying surface when the medical device is subjected to strain. For example, the coating can substantially adhere to the underlying surface when the medical device is subjected up to about 30% strain (e.g., up to about 20% strain, or up to about 10% strain). The adherence can be monitored by comparing a coated surface area of the medical device prior to and after deployment. A substantial adherence can correspond to a reduction in coated surface area of about 5% or less (e.g., about 4% or less, about 3% or less, about 2% or less, or about 1% or less) compared to the original coated surface area of the device coating. For example, referring to FIG. 5, a porous coating of only therapeutic agent (free of polymer matrix) conforms to the medical device when it is expanded, with no visible delamination. FIG. 6 shows a magnified image of a portion of the coated medical device of FIG. 5, showing the porous structure of the coating. As another example, FIG. 7 shows an expanded medical device having a well-adhered porous coating of a therapeutic agent (free of polymer matrix), and FIG. 8 is a magnified image of a high strain area (e.g., at an angled segment of a stent strut) after stent expansion showing the well-adhered porous coating.

As used herein, strain refers the deformation of a medical device during deployment (e.g., expansion). In the case of a coating, strain can refer to surface strain, which is explained, for example, in Harewood et al., Annals of Biomedical Engineering, 35(9), 2007, pp 1539-53, herein incorporated by reference in its entirety. Microscopic strain limits can be macroscopically determined by mounting a medical device (e.g., a stent) on a balloon, expanding the balloon and measuring the medical device's diameter at which the device fails (e.g., breaks or fractures). As an example, a stent can fail when points along the struts reach a surface-strain limit of around 0.3 (e.g., stents can successful expand to surface strain limits just below 0.3, or 30%). Methods of measuring stent diameter during an expansion test is described, for example, in Schmidt et al., New Aspects of in vitro Testing of Arterial Stents based on the new European Standard EN 14299, herein incorporated in its entirety.

The coating can be about 95% or more (e.g., about 96% or more, about 97% or more, about 98% or more, or about 99% or more) adherent to a medical device upon expansion or contraction. In some embodiments, the coating can be about 95% (e.g., about 97%, or about 99%) adherent to a medical device following delivery. The % adherence can be measured by comparing the coated surface areas after and before contraction or expansion of the medical device. The coated surface areas can be assessed, for example, by conducting a standard in vitro track test followed by microscopy (e.g., SEM analysis) of a medical device surface, where the micrographs are examined before and after complete expansion or contraction of a medical device.

In some embodiments, when deployed in a body lumen, coating 42 can release about 50% or more (e.g., about 60% or more, about 70% or more, about 80% or more, or about 90% or more) of the therapeutic agent in a duration of 10 days or less (e.g., eight days or less, six days or less, four days or less, or one day or less).

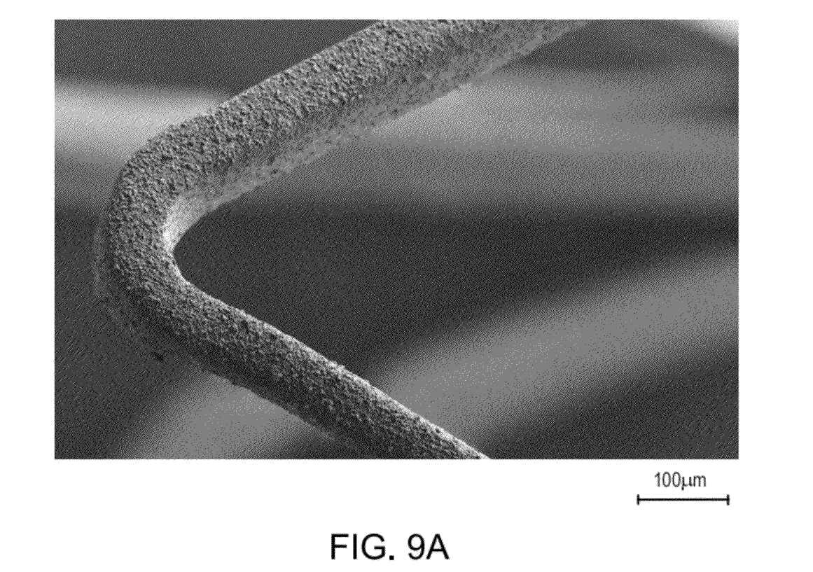

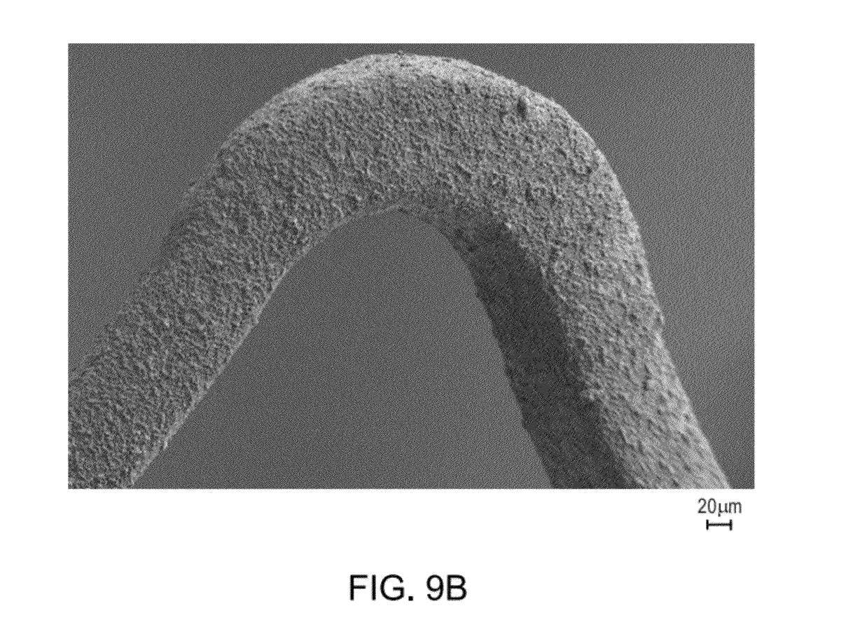

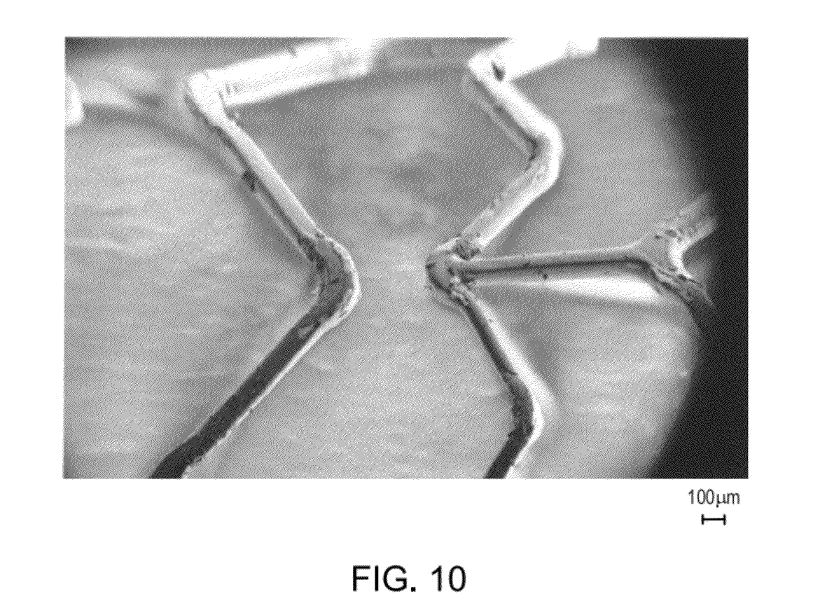

In some embodiments, coating 42 can further include an inorganic oxide layer of aluminum oxide, titanium oxide, tin oxide, and/or silica. The layer can be deposited using atomic layer deposition (ALD). The layer of aluminum oxide, titanium oxide, tin oxide, and/or silica (i.e., silicon dioxide) can infiltrate the pores within coating 42 and coat the solid surfaces on and within the coating. Referring to FIG. 9, the resulting coating can be adherent to an underlying substrate, be substantially uniform (e.g., substantially free of macrocracks, while allowing for microcracks), and be relatively durable. As used herein, a durable coating can remain intact up to deployment of the stent in an artery. The coating including an inorganic oxide can be permeable or impermeable to water. For example, an aluminum oxide coating can be permeable. In some embodiments, the coating is impermeable, such that a drug can elute when the coating dissolves or erodes, and/or the drug can elute through imperfections in the coating, such as microcracks (e.g., fissures in a porous coating that do not extend in depth to the medical device surface at greater than about 50% of the fissure length, and that extends over less than about half a strut width when the medical device is a stent. For example, the microcrack can have a length that is less than 40 micrometers) that can occur when the stent is expanded. The aluminum oxide, titanium oxide, tin oxide, and/or silica layer can mediate release of the one or more therapeutic agents in the coating.

In some embodiments, the layer of aluminum oxide, titanium oxide, tin oxide, and/or silica can have an average thickness of about 10 nm or less (e.g., about five nm or less, about three nm or less, about two nm or less, or about one nm or less) and/or about one nm or more (e.g., about two nm or more, about three nm or more, about five nm or more, or about 10 nm or more). For example, the inorganic oxide layer can have an average thickness of two nanometers. In some embodiments, the inorganic oxide layer can have an average thickness of about five nanometers. In some embodiments, thickness is measured by coating a flat substrate (such as silicon) at the same time as a stent and then using optical ellipsometry to determine the thickness deposited on the flat substrate.

In some embodiments, the layer of inorganic oxide, such as aluminum oxide, titanium oxide, tin oxide, and/or silica, is deposited by atomic layer deposition. Atomic layer deposition is described, for example, in U.S. Patent publication No. 2011/0022160, and in Heo et al., Chem. Mater., 2010, 22 (17), pp 4964-4973, herein incorporated by reference in its entirety.

The inorganic oxide layer can decrease the release rate of a therapeutic agent in the porous coating, and can increase the duration of time the porous coating can remain in a body lumen. A drug release profile can be determined by measuring drug release with and without the inorganic oxide layer. Without wishing to be bound by theory, it is believed that an effective diffusion coefficient describes diffusion through the pore space of porous media. The effective diffusion coefficient is macroscopic in nature, as an entire drug eluting coating is considered. The effective diffusion coefficient for transport through the pores, Dₑ, is estimated as follows:

$${D}_{e} = \frac{D {ɛ}_{t} δ}{τ}$$

where:

D is a diffusion coefficient in gas or liquid filling the pores (m²s⁻¹);

εₜ is porosity available for the transport (dimensionless);

δ is constrictivity (dimensionless); and

τ is tortuosity (dimensionless)

Thus, a delay in diffusion is a complex combination of the numerous factors such as porosity, constrictivity, and tortuosity, but can also depend on the dissolution of an inorganic oxide layer during the drug elution process and the thickness of the inorganic oxide layer. For a porous coating including an inorganic oxide layer, the percentage drug release can be determined by measuring a concentration of a drug in a surrounding solution by high pressure liquid chromatography at various time points. As an example, a porous coating including an inorganic oxide layer can have a percent drug release of about 90% or less (e.g., about 80% or less, about 40% or less, or about 10% or less) by weight compared to a porous coating without an inorganic oxide layer at 1 hour, a percent drug release of about 80% or less (e.g., about 60% or less, about 40% or less, or about 10% or less) by weight compared to a porous coating without an inorganic oxide layer at 24 hours, or a percent drug release of about 50% or less (e.g., about 50% or less, about 20% or less, or about 5% or less) by weight compared to a porous coating without an inorganic oxide layer at 240 hours.

In some embodiments, the porous coating can optionally include a polymer. The polymer can be biodegradable. The polymer can, for example, provide structural support for a therapeutic agent coating that may be fragile, and/or can slow the release of a therapeutic agent within the coating. The porous coating can include about 90% or less (e.g., about 75% or less, about 50% or less, about 25% or less, about 10% or less, or about 5% or less) and/or about 5% or more (e.g., about 10% or more, about 25% or more, about 50% or more, or about 75% or more) by weight of a polymer. The polymer can form a homogeneous mixture with one or more therapeutic agents within the coating, or can be a separate layer over or under the one or more therapeutic agents. In some embodiments, the polymer can form a porous network that intertwines with a porous layer of one or more therapeutic agents.

In some embodiments, a porous drug coating is made by spray coating a substrate with a solution including one or more therapeutic agents, a relatively volatile organic solvent, and a relatively less volatile solvent. In some embodiments, the one or more therapeutic agents are dissolved in one of the solvents to form a solution that is then sprayed onto a substrate, while the remaining solvent is simultaneously sprayed onto the substrate. In some embodiments, the relatively volatile organic solvent is tetrahydrofuran, methanol, acetone, chloroform, and/or other volatile solvents. The relatively less volatile solvent can include water. In some embodiments, the solution can include one or more relatively volatile organic solvent(s), and one or more relatively less volatile solvent(s). A medical device can be coated either in its expanded state, contracted state, or semi-contracted state. For example, a nitinol stent can be coated in its expanded state; a balloon expandable stent can be coated in a semi-contracted state.

For a relatively hydrophobic therapeutic agent, a porous drug coating can be made by dissolving or suspending the therapeutic agent in a mixture of a volatile organic solvent and a less volatile solvent such as water, and spraying (e.g., electrospraying) the solution onto a medical device substrate. In some embodiments, the therapeutic agent is dissolved in an organic solvent and sprayed onto the medical device using a first nozzle, and a less volatile solvent (e.g., water) is simultaneously sprayed onto the medical device using a second nozzle. In some embodiments, the solution can include one or more relatively volatile organic solvent(s), and one or more relatively less volatile solvent(s). The totality of solvents can include about 5% or more (e.g., about 10% or more, about 20% or more, about 30% or more, about 40% or more, about 50% or more, about 60% or more, about 70% or more, about 80% or more, or about 90% or more) and/or about 95% or less (e.g., about 90% or less, about 80% or less, about 70% or less, about 60% or less, about 50% or less, about 40% or less, about 30% or less, about 20% or less, or about 10% or less) by volume of one or more volatile organic solvent(s). The totality of solvents can include about 5% or more (e.g., about 10% or more, about 20% or more, about 30% or more, about 40% or more, about 50% or more, about 60% or more, about 70% or more, about 80% or more, about 90% or more) and/or about 95% or less (e.g., about 90% or less, about 80% or less, about 70% or less, about 60% or less, about 50% or less, about 40% or less, about 30% or less, about 20% or less, or about 10% or less) by volume of one or more less volatile solvent(s) (e.g., water). For example, the solvent mixture can include a about 1:1 by volume ratio of methanol to water, about 1:1 by volume ratio of acetone and water, about 8:2 by volume ratio of methanol to water, or about 1:1 by volume ratio of chloroform to water. In some embodiments, the volumetric ratio of water to volatile organic solvent(s) is about 50:50 or greater (e.g., about 60:40 or greater, about 70:30 or greater, or about 80:20 or greater) and/or about 80:20 or less (e.g., about 70:30 or less, about 60:40 or less, or about 50:50 or less). Higher water content can lead to a coating having greater porosity. Examples of hydrophobic therapeutic agents include paclitaxel, everolimus, rapamycin, sirolimus, and/or tacrolimus. In some embodiments, a hydrophilic drug can be formed into nanoparticles that are surrounded by a hydrophobic coating, such that the hydrophilic drug can behave in a similar manner as a hydrophobic drug. For example, the hydrophobic coating can be in the form of a micelle, and can include micelle-forming agents such as lecithin (a negatively charged surfactant) or stearylamine (a positively charged surfactant).

For a relatively hydrophilic therapeutic agent, a porous drug coating can be made by dissolving or suspending the therapeutic agent in a mixture of a solubilizing solvent and a miscible non-solubilizing organic solvent, and spraying (e.g., electrospraying) the solution onto a medical device substrate. For example, the relatively hydrophilic therapeutic agent can be dissolved in a solvent in which the drug is soluble (e.g., water), and sprayed onto the medical device using a first nozzle, and a second water-miscible organic solvent in which the therapeutic agent is relatively insoluble is simultaneous sprayed onto the medical device using a second nozzle. The water-miscible organic solvent can have a higher boiling point than water and can include, for example, ethylene glycol, propylene glycol, and mixtures thereof. In some embodiments, the solution can include one or more solubilizing solvent(s), and one or more miscible non-solubilizing solvent(s) having a higher boiling point than the one or more solubilizing solvent(s).

In some embodiments, a relatively hydrophilic therapeutic agent is dissolved or suspended in an emulsion of immiscible solvents. The emulsion of solvents can include a solvent in which the therapeutic agent is soluble, and an immiscible solvent in which the therapeutic agent is relatively insoluble. The solvents can be agitated such that one solvent is suspended in the other to form an emulsion during the coating process. The immiscible solvent can have a higher boiling point than the solvent in which the hydrophilic therapeutic agent is soluble. For example, the immiscible solvent can include butyl acetate, and the solubilizing solvent can include water. In some embodiments, the emulsion can include one or more solubilizing solvent(s), and one or more immiscible non-solubilizing solvent(s) having a higher boiling point than the one or more solubilizing solvent(s).

For a relatively hydrophilic therapeutic agent, the solvent mixture can include about 5% or more (e.g., about 10% or more, about 20% or more, about 30% or more, about 40% or more, about 50% or more, about 60% or more, about 70% or more, about 80% or more, about 90% or more) and/or about 95% or less (e.g., about 90% or less, about 80% or less, about 70% or less, about 60% or less, about 50% or less, about 40% or less, about 30% or less, about 20% or less, or about 10% or less) by volume of the one or more solubilizing solvent(s) (e.g., water). The solvent mixture can include about 5% or more (e.g., about 10% or more, about 20% or more, about 30% or more, about 40% or more, about 50% or more, about 60% or more, about 70% or more, about 80% or more, about 90% or more) and/or about 95% or less (e.g., about 90% or less, about 80% or less, about 70% or less, about 60% or less, about 50% or less, about 40% or less, about 30% or less, about 20% or less, or about 10% or less) by volume of one or more non-solubilizing solvent(s) having a higher boiling point than the solubilizing solvent(s). In some embodiments, the solvent mixture can include about 4:1 by volume ratio of water to ethylene glycol; or about 1:1 by volume ratio of water to butyl acetate. In some embodiments, the volumetric ratio of solubilizing solvent(s) to non-solubilizing solvent(s) is about 50:50 or greater (e.g., about 60:40 or greater, about 70:30 or greater, or about 80:20 or greater) and/or about 80:20 or less (e.g., about 70:30 or less, about 60:40 or less, or about 50:50 or less). Examples of hydrophilic therapeutic agents include heparin, diclofenac, and aspirin.

In some embodiments, both hydrophobic and hydrophilic therapeutic agents can be coated onto a medical device as a porous coating. For example, the porous drug coating can be made by forming a first solution of a hydrophobic therapeutic agent in a volatile organic solvent and a second solution of a hydrophilic therapeutic agent in water, and simultaneously spraying (e.g., electrospraying) the first solution from a first nozzle and a second solution from a second nozzle onto a medical device substrate. As another example, the porous coating can be made by forming a first solution of a hydrophobic therapeutic agent in miscible or immiscible organic solvent having a higher boiling point than water, and a second solution of a hydrophilic therapeutic agent in water, and simultaneously spraying (e.g., electrospraying) the first solution from a first nozzle and a second solution from a second nozzle onto a medical device substrate. In some embodiments, the two solutions can be mixed together and sprayed onto a medical device using a single nozzle. The solvents can include about 5% or more (e.g., about 10% or more, about 20% or more, about 30% or more, about 40% or more, about 50% or more, about 60% or more, about 70% or more, about 80% or more, about 90% or more) and/or about 95% or less (e.g., about 90% or less, about 80% or less, about 70% or less, about 60% or less, about 50% or less, about 40% or less, about 30% or less, about 20% or less, or about 10% or less) by volume of water. The solvents can include about 5% or more (e.g., about 10% or more, about 20% or more, about 30% or more, about 40% or more, about 50% or more, about 60% or more, about 70% or more, about 80% or more, about 90% or more) and/or about 95% or less (e.g., about 90% or less, about 80% or less, about 70% or less, about 60% or less, about 50% or less, about 40% or less, about 30% or less, about 20% or less, or about 10% or less) by volume of one or more volatile organic solvent(s), or one or more organic solvent(s) having a higher boiling point than water. In some embodiments, the solvents can include about 1:1 by volume ratio of methanol to water, about 1:1 by volume ratio of acetone to water, about 1:1 by volume ratio of chloroform to water, about 2:8 by volume ratio of methanol to water, about 4:1 by volume ratio of water to ethylene glycol; or about 1:1 by volume ratio of water to butyl acetate. In some embodiments, the volumetric ratio of water to volatile organic solvent(s) or higher boiling point organic solvent(s) is about 50:50 or greater (e.g., about 60:40 or greater, about 70:30 or greater, or about 80:20 or greater) and/or about 80:20 or less (e.g., about 70:30 or less, about 60:40 or less, or about 50:50 or less). In some embodiments, the volumetric ratio of water to volatile organic solvent(s) is about 50:50.

The therapeutic agent can be dissolved in a solution at a w/w (weight/weight) concentration of about 1% or more (e.g., about 5% or more, about 10% or more, or about 15% or more) and/or about 20% or less (e.g., about 15% or less, about 10% or less, or about 5% or less) relative to a total solution volume, which includes the volumes of volatile organic solvent and water. The therapeutic agent concentration can vary throughout a coating process to provide a medical device having a variable drug release profile. For example, the therapeutic agent concentration can be greater when coating the surface of a coated medical device than when coating near the immediate surface of the medical device substrate, such that the resulting medical device can release a greater amount of therapeutic agent immediately after deployment. As another example, the therapeutic agent concentration can be smaller near the surface of a coated medical device than near the immediate surface of the medical device substrate. In some embodiments, one or more therapeutic agents can be coated onto a medical device, each at different concentrations. The concentration of each of the therapeutic agent can vary throughout the medical device coating.

After spraying the medical device with one or more solutions as described above, the solvents are evaporated to form a porous structure. The solvent can evaporate under ambient pressure (i.e., 1 atm), at reduced pressures, at ambient temperature (i.e., 21° C.), at lower temperatures or at higher temperatures than ambient temperature. A higher spraying and/or evaporation temperature can lead to smaller pores and a denser coating. Without wishing to be bound by theory, it is believed that a porous coating results when a volatile organic solvent containing a drug evaporates while avoiding slower evaporating water regions, to provide a porous drug framework including water within the pores. The water eventually evaporates, leaving the pores. Similarly, a porous coating can result when a solvent containing a hydrophilic drug evaporates while avoiding slower evaporating organic solvent regions.

In some embodiments, certain therapeutic agents, bioabsorbable polymers, or other components of the porous coating are susceptible to hydrolysis. Therefore, a solution containing water-sensitive components can be formed immediately prior to coating, and/or can be sprayed from a separate nozzle than a water nozzle, to minimize degradation of the water-sensitive components.

In some embodiments, the solution of one or more therapeutic agents can further include a pharmaceutically acceptable carrier. Suitable pharmaceutically-acceptable carriers are known in the art; for example, see Remington, The Science and Practice of Pharmacy, 20th Edition, 2000, Lippincott Williams & Wilkins, (Editors: Gennaro, A. R., et al.).

In some embodiments, in addition to spraying a solution of one or more therapeutic agents, a polymer solution can also be sprayed onto the medical device. The polymer solution can be simultaneously sprayed onto the medical device, or sprayed onto the medical device before or after coating the device with one or more therapeutic agents. A porous polymer coating can be formed by dissolving a polymer in a mixture of a volatile organic solvent(s) and water, and spraying (e.g., electrospraying) the solution onto a medical device substrate. In some embodiments, the polymer can be dissolved in a solution containing one or more therapeutic agents. The polymer can be dissolved in one or more volatile organic solvent(s) or water, and sprayed onto the medical device using a first nozzle; while water (if the polymer is dissolved in an organic solvent) or a volatile organic solvent (if the polymer is dissolved in water) is simultaneous sprayed onto the medical device using a second nozzle. The solvents can include about 5% or more (e.g., about 10% or more, about 20% or more, about 30% or more, about 40% or more, about 50% or more, about 60% or more, about 70% or more, about 80% or more, about 90% or more) and/or about 95% or less (e.g., about 90% or less, about 80% or less, about 70% or less, about 60% or less, about 50% or less, about 40% or less, about 30% or less, about 20% or less, or about 10% or less) by volume of water. The solvents can include about 5% or more (e.g., about 10% or more, about 20% or more, about 30% or more, about 40% or more, about 50% or more, about 60% or more, about 70% or more, about 80% or more, or about 90% or more) and/or about 95% or less (e.g., about 90% or less, about 80% or less, about 70% or less, about 60% or less, about 50% or less, about 40% or less, about 30% or less, about 20% or less, or about 10% or less) by volume of one or more volatile organic solvent(s). In some embodiments, the solvents can include a about 1:1 by volume ratio of methanol to water, about 1:1 by volume ratio of acetone to water, about 1:1 by volume ratio of chloroform to water, about 2:8 by volume ratio of methanol to water, 4:1 by volume ratio of water to ethylene glycol; or about 1:1 by volume ratio of water to butyl acetate. In some embodiments, the volumetric ratio of water to volatile organic solvent(s) is about 50:50 or greater (e.g., about 60:40 or greater, about 70:30 or greater, or about 80:20 or greater) and/or about 80:20 or less (e.g., about 70:30 or less, about 60:40 or less, or about 50:50 or less). In some embodiments, the volumetric ratio of water to volatile organic solvent(s) is about 50:50.

The polymer can have a w/w concentration of about 1% or more (e.g., about 3% or more, about 5% or more, about 10% or more, or about 15% or more) and/or about 20% or less (e.g., about 15% or less, about 10% or less, about 5% or less, or about 3% or less) relative to a total polymer solution volume, which includes the volumes of volatile organic solvent(s) and water. The polymer concentration can vary throughout a coating process. For example, the polymer concentration can be smaller when coating near the surface of a coated medical device than when coating near the immediate surface of the medical device substrate, or the polymer concentration can be greater when coating near the surface of a coated medical device than when coating near the immediate surface of the medical device substrate. In some embodiments, one or more polymers can be coated onto a medical device, each at different concentrations. The concentration of each of the polymers can vary throughout the medical device coating. Examples of polymers include without limitation polyurethane and its copolymers, silicone and its copolymers, ethylene vinyl-acetate, polyethylene terephthalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polycarbonate, poly(glycolide-lactide) copolymer, polylactic acid, poly(y-caprolactone), poly(y-hydroxybutyrate), polydioxanone, poly(y-ethyl glutamate), polyiminocarbonates, poly(ortho ester), polyanhydrides, alginate, dextran, chitin, cotton, polyglycolic acid, polylactic acid-polyethylene oxide copolymers, cellulose, collagens, and chitins.

In some embodiments, an inorganic oxide layer can be deposited using atomic layer deposition. The inorganic oxide layer can include aluminum oxide, titanium oxide, tin oxide, and/or silica. The inorganic oxide layer can be applied before, or after deposition of the porous therapeutic agent layer and/or the polymer layer. In some embodiments, a combination of coating methods can be used to deposit various polymers, inorganic oxides, or therapeutic agents, in addition to the deposition methods described above. For example, additional polymer, inorganic oxides, or therapeutic agents can be coated onto the medical device using methods such as conventional nozzle or ultrasonic nozzle spraying, dipping, rolling, electrostatic deposition, and a batch process such as air suspension, pancoating or ultrasonic mist spraying. As an example, in some embodiment, a first porous coating including a first therapeutic agent can be applied to a medical device substrate by the method described above. A second layer of an inorganic oxide can then be applied using atomic layer deposition, then a third layer of a second therapeutic agent can be applied by dip-coating the medical device into a solution of the second therapeutic agent in a solvent. The first porous coating including the inorganic oxide layer can serve as a scaffold for the dip-coated second therapeutic agent. The dip-coating can be relatively rapid, to preserve the porous structure an underlying coating. As another example, a first porous coating including a first therapeutic agent and a first polymer can be applied to a medical device substrate by the method described above. A second permeable or impermeable layer of an inorganic oxide can then be applied using atomic layer deposition, and then a second therapeutic agent can be applied to the medical device using the porous coating method described above. As a further example, a first porous polymer coating can be deposited on a medical device substrate by the method described above. An inorganic oxide layer can be applied to the porous polymer coating using atomic layer deposition, then a therapeutic agent can be deposited onto the porous polymer scaffold by dip-coating. When an inorganic layer is coated onto a medical device before the one or more therapeutic agents, the inorganic layer can include transition oxides that can be deposited at higher temperatures using atomic layer deposition, such as tantalum oxide, iridium oxide, and/or ruthenium oxide.

In some embodiments, a polymeric porous coating can be deposited onto a medical device that is formed of the same polymer as that in the coating. A porous therapeutic agent layer can be deposited by forming a solution of the therapeutic agent in a volatile organic solvent and water. The solution can be homogenized using ultrasonic means to make a fine dispersion of nanosized water droplets in the organic solvent. The porous polymer coated medical device can be dip-coated in the homogenized therapeutic agent dispersion. The solvents are then evaporated to deposit the therapeutic agent within and over the porous polymer coating. An inorganic oxide layer can optionally be deposited using atomic layer deposition. The resulting medical device can be considered to have a porous drug within the device, as the porous polymer layer is composed of the same polymer as the medical device substrate.

In some embodiments, it may be desirable to roughen a surface of interest before performing depositions described herein. For example, a surface may be roughened to provide a series of nooks or invaginations on/within the surface. Any surface may be roughened, e.g., a metallic, polymeric or ceramic surface. Surfaces can be roughened using any technique known in the art. Particularly useful methods for roughening surfaces, such as the surfaces of a stent, are described, e.g., in U.S. Ser. No. 12/205,004, which is hereby incorporated by reference. The surface of a balloon may also be roughened.

Further, as will be appreciated by skilled practitioners, coatings described herein can be deposited on an entire surface of a device or onto only part of a surface. This can be accomplished using masks to shield the portions on which coatings are not to be deposited. Further, with regard to stents, it may be desirable to deposit only on the abluminal surface of the stent. This construction may be accomplished by e.g. coating the stent before forming the fenestrations. In other embodiments, it may be desirable to deposit only on abluminal and cutface surfaces of the stent. This construction may be accomplished by, e.g., depositing on a stent containing a mandrel, which shields the luminal surfaces.

The terms “therapeutic agent”, “pharmaceutically active agent”, “pharmaceutically active material”, “pharmaceutically active ingredient”, “drug” and other related terms may be used interchangeably herein and include, but are not limited to, small organic molecules, peptides, oligopeptides, proteins, nucleic acids, oligonucleotides, genetic therapeutic agents, non-genetic therapeutic agents, vectors for delivery of genetic therapeutic agents, cells, and therapeutic agents identified as candidates for vascular treatment regimens, for example, as agents that reduce or inhibit restenosis. By small organic molecule is meant an organic molecule having 50 or fewer carbon atoms, and fewer than 100 non-hydrogen atoms in total. Generally, exemplary therapeutic agents include, e.g., sirolimus, everolimus, biolimus, zotarolimus, tacrolimus and paclitaxel. The therapeutic agent can be amorphous.

Exemplary non-genetic therapeutic agents include anti-thrombogenic agents such as heparin, heparin derivatives, prostaglandin (including micellar prostaglandin E1), urokinase, and PPack (dextrophenylalanine proline arginine chloromethylketone); anti-proliferative agents such as enoxaparin and angiopeptin, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; antimicrobial agents such as triclosan, cephalosporins, aminoglycosides, nitrofurantoin, silver ions, compounds, or salts; biofilm synthesis inhibitors such as non-steroidal anti-inflammatory agents and chelating agents such as thylenediaminetetraacetic acid, O,O′-bis (2-aminoethyl)ethyleneglycol-N,N,N′,N′-tetraacetic acid and mixtures thereof; antibiotics such as gentamycin, rifampin, minocyclin, and ciprofloxacin; antibodies including chimeric antibodies and antibody fragments; anesthetic agents such as lidocaine, bupivacaine, and ropivacaine; nitric oxide; nitric oxide (NO) donors such as linsidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; inhibitors of heat shock proteins such as geldanamycin; angiotensin converting enzyme (ACE) inhibitors; beta-blockers; βAR kinase (βARK) inhibitors; phospholamban inhibitors; proteinbound particle drugs such as ABRAXANE™; structural protein (e.g., collagen) cross-link breakers such as alagebrium (ALT-711); and/or any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (MCP-1) and bone morphogenic proteins (“BMPs”), such as, for example, BMP-2, BMP-3, BMP-4, BMP-5, BMP-6 (VGR-1), BMP-7 (OP-1), BMP-8, BMP-9, BMP-10, BMP-11, BMP-12, BMP-13, BMP-14, and BMP-15. Preferred BMPs are any of BMP-2, BMP-3, BMP-4, BMP-5, BMP-6, and BMP-7. These BMPs can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNAs encoding them. Non-limiting examples of genes include survival genes that protect against cell death, such as antiapoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factors α and β, platelet-derived endothelial growth factor, platelet-derived growth factor, tumor necrosis factor α, hepatocyte growth factor, and insulin-like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase and combinations thereof and other agents useful for interfering with cell proliferation.

Exemplary small molecules include hormones, nucleotides, amino acids, sugars, and lipids and compounds having a molecular weight of less than 100 kD.

Suitable medical devices include, but are not limited to, those that have a tubular or cylindrical like portion. A tubular portion of a medical device need not be completely cylindrical. The cross-section of the tubular portion can be any shape, such as rectangle, a triangle, etc., not just a circle. Such devices include, but are not limited to, stents, balloons of a balloon catheters, grafts, and valves (e.g., a percutaneous valve). A bifurcated stent is also included among the medical devices which can be fabricated by the methods described herein. The device can be made of any material, e.g., metallic, polymeric, and/or ceramic material.

The stents described herein can be configured for vascular, e.g. coronary and peripheral vasculature or non-vascular lumens. For example, they can be configured for use in the esophagus or the prostate. Other lumens include biliary lumens, hepatic lumens, pancreatic lumens, uretheral lumens and ureteral lumens.

Any stent described herein can be dyed or rendered radiopaque by addition of, e.g., radiopaque materials such as barium sulfate, platinum or gold, or by coating with a radiopaque material. The stent can include (e.g., be manufactured from) metallic materials, such as stainless steel (e.g., 316L, BioDur® 108 (UNS S29108), and 304L stainless steel, and an alloy including stainless steel and 5-60% by weight of one or more radiopaque elements (e.g., Pt, Ir, Au, W) (PERSS®) as described in US-2003-0018380-A1, US-2002-0144757-A1, and US-2003-0077200-A1), Nitinol (a nickel-titanium alloy), cobalt alloys such as Elgiloy, L605 alloys, MP35N, titanium, titanium alloys (e.g., Ti-6Al-4V, Ti-50Ta, Ti-10Ir), platinum, platinum alloys, niobium, niobium alloys (e.g., Nb-1Zr) Co-28Cr-6Mo, tantalum, and tantalum alloys. Other examples of materials are described in commonly assigned U.S. application Ser. No. 10/672,891, filed Sep. 26, 2003; and U.S. application Ser. No. 11/035,316, filed Jan. 3, 2005. Other materials include elastic biocompatible metal such as a superelastic or pseudo-elastic metal alloy, as described, for example, in Schetsky, L. McDonald, “Shape Memory Alloys”, Encyclopedia of Chemical Technology (3rd ed.), John Wiley & Sons, 1982, vol. 20. pp. 726-736; and commonly assigned U.S. application Ser. No. 10/346,487, filed Jan. 17, 2003.

A stent can be of a desired shape and size (e.g., coronary stents, aortic stents, peripheral vascular stents, gastrointestinal stents, urology stents, tracheal/bronchial stents, and neurology stents). Depending on the application, the stent can have a diameter of between, e.g., about 1 mm to about 46 mm. In certain embodiments, a coronary stent can have an expanded diameter of from about 2 mm to about 6 mm. In some embodiments, a peripheral stent can have an expanded diameter of from about 4 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. The stent can be balloon-expandable, self-expandable, or a combination of both (e.g., U.S. Pat. No. 6,290,721). The ceramics can be used with other endoprostheses or medical devices, such as catheters, guide wires, and filters.

### EXAMPLES

### Example 1

A solution was made up with the following w/w proportions:

| | |
|---|---|
| Everolimus | 3.5% |
| Methanol | 35% |
| Acetone | 23.5% |
| Water | 38% |

The everolimus was dissolved in the methanol and acetone first, and then the water was gradually added. The solution was sprayed on a stent using a standard gas assist atomization nozzle with Nitrogen pressure of 20 psi and a solution flow rate of about 20 ml/hr until a porous coating on the stent of approximately 5-10 μg/mm (i.e., the coat weight per mm length of the stent) was obtained. The stent was tested by crimping on a balloon, immersing in deionized water for 2 minutes, removing from the water and immediately expanding using the balloon. The stent was then dried in air and inspected using an SEM. Referring to FIGS. 7, 9A, and 9B, the coating was found to be intact. In comparison, referring to FIG. 10, a solid coating of the same weight per mm showed significant delamination.

### Example 2

A solution was made up with the following w/w proportions:

| | |
|---|---|
| Everolimus | 3.5% |
| Cyclohexanone | 19.5% |
| Acetone | 44% |
| Water | 33% |

The everolimus was dissolved in the acetone and cyclohexanone first and then the water was gradually added. The solution was sprayed on a stent using a standard gas assist atomization nozzle with Nitrogen pressure of 20 psi and a solution flow rate of about 20 ml/hr until a porous coating on the stent of approximately 5-10 μg/mm (i.e the coat weight per mm length of the stent) was obtained. The stent was tested by crimping on a balloon, immersing in deionized water for 2 minutes, removing from the water and immediately expanding using the balloon. The stent was then dried in air and inspected using an SEM. The coating was found to be intact. In comparison, referring to FIG. 10, a solid coating of the same weight per mm showed significant delamination.

### Example 3

A solution was made of 4% w/w paclitaxel, 86.4% w/w cyclohexanone and 9.6% w/w tetrahydrofuran. The solution was sprayed on a stent using a standard gas assist atomization nozzle with Nitrogen pressure of 20 psi and a solution flow rate of 20 ml/hr until a coating on the stent of approximately 5-10 μg/mm (i.e the coat weight per mm length of the stent) was obtained. A SEM micrograph of the coated stent shows delamination.

### Example 4

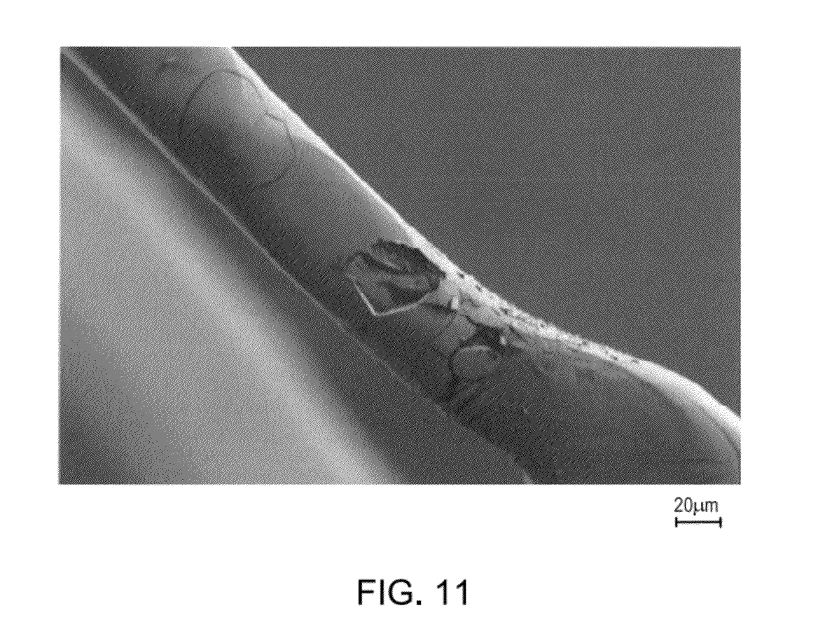

A solution was made of 4% w/w everolimus and 96% w/w butyl acetate. The solution was coated on a stent using a inkjet nozzle with a 30 μm orifice until a coating of approximately 5-10 μg/mm (i.e., the coat weight per mm length of the stent) was obtained. A SEM micrograph of the coated stent is shown in FIG. 11, where significant delamination of the solid coating is depicted.

The foregoing description and examples have been set forth merely to illustrate the disclosure and are not intended to be limiting. Each of the disclosed aspects and embodiments of the present disclosure may be considered individually or in combination with other aspects, embodiments, and variations of the disclosure. Modifications of the disclosed embodiments incorporating the spirit and substance of the disclosure may occur to persons skilled in the art and such modifications are within the scope of the present disclosure.

What is claimed is:

1. An expandable medical device comprising a porous substantially polymer-free coating comprising a therapeutic agent, wherein the coating substantially adheres to the medical device upon expansion of the medical device.

2. The expandable medical device of claim 1, wherein the coating further comprises aluminum oxide, titanium oxide, tin oxide, zinc oxide, or silica.

3. The expandable medical device of claim 1, wherein the coating has a porosity of about 20% or more.

4. The expandable medical device of claim 1, wherein the porous substantially polymer-free coating consists essentially of one or more therapeutic agents.

5. The expandable medical device of claim 1, wherein the expandable medical device comprises a stent, a balloon, and a balloon catheter.

6. The expandable medical device of claim 1, wherein the expandable medical device comprises a self-expanding stent and a delivery catheter.

7. The expandable medical device of claim 1, wherein the therapeutic agent is selected from the group consisting of paclitaxel, everolimus, rapamycin, sirolimus, tacrolimus, heparin, diclofenac, aspirin, and any combination thereof.

8. The expandable medical device of claim 1, wherein the therapeutic agent is amorphous.

9. The expandable medical device of claim 1, wherein the coating is more than about 95% adherent to the medical device upon expansion or contraction.

10. The expandable medical device of claim 1, wherein when inserted to a predetermined location in a blood vessel, about 50% or more of the therapeutic agent is released from the coating in 10 days or less.

11. A method of making a medical device, comprising:
step (a): forming a mixture comprising a therapeutic agent, an organic solvent, and optionally water;
step (b): providing a solution comprising water, when the mixture in step (a) is water-free;
step (c): coating the medical device with the mixture and the solution, when present; and
step (d): evaporating the organic solvent and water to provide a porous coating comprising a therapeutic agent.

12. The method of claim 11, wherein step (c) further comprises simultaneously coating the medical device with the mixture and the solution, when present.

13. The method of claim 11, wherein prior to evaporation, the ratio of organic solvent to water on the medical device is about 1:1 or greater.

14. The method of claim 11, wherein coating the medical device comprises spraying the medical device.

15. The method of claim 11, wherein the mixture further comprises a polymer.

16. The method of claim 11, wherein the solution further comprises a polymer.

17. The method of claim 11, further comprising step (e): coating the medical device with aluminum oxide, titanium oxide, tin oxide, zinc oxide, silica, or combinations thereof.

18. The method of claim 17, wherein in step (e), coating the medical device comprises atomic layer deposition.

19. The method of claim 17, wherein step (e) precedes step (c) or follows step (d).

20. The method of claim 17, further comprising repeating one or more of steps (a), (b), (c), (d), or (e).

21. The method of claim 11, wherein the porous coating substantially adheres to the medical device upon expansion of the medical device.

22. The method of claim 11, wherein when the therapeutic agent is hydrophobic.

23. The method of claim 11, wherein the therapeutic agent is selected from the group consisting of paclitaxel, everolimus, rapamycin, sirolimus, tacrolimus, and any combination thereof.

24. A method of making a medical device, comprising:
step (a): forming a mixture comprising a hydrophilic therapeutic agent and water;
step (b): providing a solution comprising a solvent having a higher boiling point than water;
step (c): coating the medical device with the mixture and the solution; and
step (d): evaporating the water and solution to provide a porous coating comprising a hydrophilic therapeutic agent.

25. The method of claim 24, wherein step (c) further comprises simultaneously coating the medical device with the mixture and the solution.

26. The method of claim 24, wherein the hydrophilic therapeutic agent comprises heparin, diclofenac, and aspirin.

27. A method of making a medical device, comprising:
step (a): forming a mixture comprising a therapeutic agent, an organic solvent, and water;
step (b): ultrasonicating the mixture to provide a dispersion;
step (c): coating the medical device with the dispersion; and
step (d): evaporating the water and organic to provide a porous coating comprising a therapeutic agent.

28. The method of claim 27, wherein coating comprises dip-coating and spray-coating.

29. The method of claim 27, further comprising step (e) before step a: applying a porous polymer coating onto the medical device.

30. The method of claim 29, further comprising step (f) after step (e) and before step (a), or after step (d): coating the medical device with aluminum oxide, titanium oxide, tin oxide, zinc oxide, silica, or combinations thereof.
